(19) United States
(12) Patent Application Publication
(10) Pub. No.: US 2026/0295107 A1
(43) Pub. Date: Oct. 1, 2026

(54) DEPLOYABLE NON-POWERED FRAGRANCE DIFFUSER

(71) Applicant: Votivo, LLC, Spartanburg, SC (US)

(72) Inventors: Robert E. Caldwell, JR., Spartanburg, SC (US); Lauren Michelle Gossett Marshall, Spartanburg, SC (US); Logan Clendenin Mode, Moore, SC (US)

(21) Appl. No.: 19/634,795

(22) Filed: Mar. 31, 2026

Related U.S. Application Data
(60) Provisional application No. 63/781,608, filed on Apr. 1, 2025.

(51) Int. Cl. A61L 9/12

(52) U.S. Cl. CPC A61L 9/127; A61L 2209/133

(57) ABSTRACT

A non-powered flameless diffuser device for ambient dispersion of fragrance includes a container holding a fragranced oil or solution and a deployable insert. The deployable insert comprises a foldable, honeycombed-style body formed from a paper or paper-like material. The insert is configured to transition from a packaged, folded flat position into a deployed, fanned-out position for use. In the deployed position, the insert includes a lower extending region that projects into the container to draw or wick the fragranced oil via capillary action. An upper diffusing region expands radially to provide an expanded surface area of folded surfaces for ambient evaporation. A recessive section is formed in some embodiments between the lower extending region and the upper diffusing region and defines a geometric region shaped to receive an upper portion of the container, seating the upper diffusing region above the container while suspending the lower extending region.

### PRIORITY CLAIM

The present application claims the benefit of priority of U.S. Provisional Patent Application No. 63/781,608, titled Honeycomb Topper Diffuser, filed Apr. 1, 2025, and which is fully incorporated herein by reference for all purposes.

### BACKGROUND OF THE PRESENTLY DISCLOSED SUBJECT MATTER

The presently disclosed subject matter relates to improved devices and methodologies for diffuser systems for the ambient dispersion of fragrance.

Reed diffusers are typically home-use fragrance products which disperse scents into the surrounding air, through the use of reeds. Without using any open flame as used in scented candles or a powered device such as a plug-in or other device that relies upon heat to accelerate the dispersion of fragrance, they spread scent via the reeds or reed sticks through a natural process referred to as capillary action that relies upon ambient evaporation and dispersion of the aromatic molecules.

Typically, a mixture of scented oils, such as essential oils or synthetic aromatic materials, are stored in a bottle or container having an upper opening. In some instances, the fragrance oil or essential oil may be mixed with a base solution. One or more reeds may be placed into the container with the oil, and so as to come into contact with the solution or oil. The reeds absorb the oil, which then evaporates into the air to release the scent. In other words, the mixture is absorbed into the sticks or reeds through capillary action, and then diffuses into the air through evaporation, to provide a room or area the associated fragrance.

Since a natural process is involved, no aerosols, heating, or burning of products is required to generate and disperse scents, so reed diffusers can be a preferred mode of scent creation in some environments or situations.

Reed sticks are typically comprised of long, thin strips of materials which are put into the associated container. Various porous materials have been practiced, including such as rattan wood, synthetic polyamide, fiber or bamboo reeds, or unglazed ceramic.

To keep the fragrance strong, it may be necessary to manage the reeds, such as by using a plurality of reeds, or flipping the reeds from time to time, or even entirely changing the reeds periodically, as the reeds sometimes become clogged with solid or resinous heavier molecular weight aromatic materials, such as vanillin, a common ingredient or component present in many fragrance formulas.

Managing the reeds may be necessary because the pathways for oil absorption and dispersion can become partially loaded or blocked over time, and fresh pathways are needed to maintain desired fragrance dispersion.

### SUMMARY OF THE PRESENTLY DISCLOSED SUBJECT MATTER

The presently disclosed subject matter relates to improved devices and methodologies for diffuser systems for the dispersion of fragrance. In particular, the present disclosed subject matter involves different embodiments using various combinations of materials and features to provide a non-powered diffuser-type device providing strong, dependable fragrance dispersion performance.

In some presently disclosed exemplary embodiments, various folded and/or honeycombed style devices may be used as inserts to containers of fragranced oil, in place of conventional reeds of a reed diffuser. While the presently disclosed inserts or toppers may be folded for packaging and/or shipping purposes, they each have unfolded or deployed positions into which they may be moved for use.

Further, per some presently disclosed exemplary embodiments, the topper devices provide significant areas of surface put into contact with the stored fragrance oil, to facilitate the solution or oil being drawn into the topper for dispersion. Additionally, the topper devices may provide significant areas of surface put into contact with the surrounding air, so that stored fragrance oil drawn into the devices may be evaporated and dispersed with high efficiency.

Still further, such foldable, deployable toppers may be formed from various types of paper or paper-like materials, to be both lightweight but also highly effective at drawing or wicking and dispersing the fragranced oil solutions.

While traditional reed diffusers may rely on rigid, thick materials such as rattan or bamboo, such conventional wicking elements suffer from limited surface area and pathways that frequently become clogged with heavier molecular weight aromatic materials over time. This may lead to a diminished fragrance “throw” and require the user to constantly manage or replace the reeds. Furthermore, packaging and shipping rigid diffusion elements requires significant commercial volume.

The presently disclosed subject matter overcomes such drawbacks by completely decoupling the wicking mechanism from traditional rigid formats. By utilizing a deployable, paper-based structure, the presently disclosed devices drastically increase the available evaporative surface area compared to traditional reeds, resulting in highly efficient capillary action and an unexpectedly strong, dependable dispersion of fragrance.

Furthermore, it is a specific object of the presently disclosed subject matter to provide an insert that dynamically adapts to containers of varying neck heights and opening diameters. The functional geometry of the disclosed inserts, particularly for embodiment with the provision of a recessive section or seating notch, uniquely allows the top-heavy, expanded diffusing region to be securely supported above the container while safely suspending the lower wicking region in the fragranced oil. This unique geometry and/or other presently disclosed aspects allows the entire high-performance device to be shipped in a compact, folded-flat configuration and easily deployed by the end user, representing a significant commercial and packaging advantage.

Finally, as used herein, terms such as “honeycomb,” “honeycombed style,” and “fanned-out” are used broadly to describe structures having a plurality of folded, cellular, or intersecting surfaces that provide an expanded surface area for evaporation, and are not strictly limited to perfect hexagonal or other geometric arrays. Similarly, terms describing spatial relationships such as “above,” “lower,” “recessive,” and “projecting” are intended to describe the relative functional positioning of the elements when deployed for use with a container, rather than absolute structural limitations.

One exemplary embodiment of presently disclosed subject matter relates to a non-powered diffuser device for the ambient dispersion of fragrance. Such device preferably may comprise a container defining an interior volume holding a fragranced oil or a fragranced solution, the container having an upper portion defining an opening; and a deployable insert positioned at least partially within the container, the deployable insert comprising a foldable honeycombed style body formed from a paper or paper-like material and configured to transition from a packaged folded flat position into a deployed fanned-out position. Preferably, in the deployed fanned-out position, the deployable insert comprises a lower extending region projecting downwardly through the opening and into the fragranced oil or a fragranced solution within the container interior volume to draw or wick the fragranced oil or a fragranced solution via capillary action; and an upper diffusing region expanding radially outward relative to a longitudinal axis of the deployable insert, the upper diffusing region comprising a plurality of folded surfaces providing an expanded surface area for ambient evaporation of the wicked fragranced oil or a fragranced solution.

Yet another exemplary embodiment in accordance with the presently disclosed subject matter may relate to a non-powered diffuser device for the ambient dispersion of fragrance, comprising a container defining an interior volume holding a fragranced oil or a fragranced solution, the container having an upper facing side wall area and defining an opening; and an insert comprising a lower wicking region received through the opening into the interior volume to draw or wick the fragranced oil or a fragranced solution via capillary action, and an upper diffusing region extending above the container, the insert comprising a foldable honeycombed style body formed from a paper or paper-like material. Such device may further preferably comprise a cover positioned over the upper diffusing region of the insert, the cover having a bottom resting on the upper facing side wall area of the container. Further preferably in some such embodiments, the cover may define a plurality of openings permitting ambient dispersion of the fragranced oil or a fragranced solution evaporated from the upper diffusing region.

It is to be understood that the presently disclosed subject matter equally relates to both disclosed devices and associated and/or corresponding methodologies.

One exemplary method of presently disclosed subject matter may preferably relate to a method for providing ambient dispersion of a fragrance. Such method preferably may comprise providing a container holding a fragranced oil or a fragranced solution, the container having an upper portion defining an opening; providing a deployable insert in a packaged folded flat position, the deployable insert comprising a foldable honeycombed style body formed from a paper or paper-like material; unfolding the deployable insert from the packaged folded flat position into a deployed fanned-out position; positioning the deployable insert at least partially into the container such that a lower extending region of the deployable insert projects downwardly through the opening and into the fragranced oil or a fragranced solution, while an upper diffusing region of the deployable insert projects upwardly above the opening and provides an expanded surface area of folded surfaces for ambient evaporation of the fragranced oil or a fragranced solution; and drawing or wicking the fragranced oil or a fragranced solution upward through the lower extending region and into the upper diffusing region through capillary action.

Additional objects and advantages of the presently disclosed subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments, uses, and practices of the presently disclosed subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the presently disclosed subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the presently disclosed subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification, and will appreciate that the presently disclosed subject matter applies equally to corresponding methodologies as associated with practice of any of the present exemplary devices, and vice versa.

These and other features, aspects and advantages of various embodiments will become better understood with reference to the following description and appended claims. The accompanying drawings, which are incorporated in and constitute a part of this specification, illustrate embodiments of the present disclosure and, together with the description, serve to explain the related principles.

### BRIEF DESCRIPTION OF THE FIGURES

A full and enabling disclosure of the present subject matter, including the best mode thereof to one of ordinary skill in the art, is set forth more particularly in the remainder of the specification, including reference to the accompanying figures in which:

FIG. 1 is an image representing an exemplary embodiment of a presently disclosed non-powered diffuser-type device comprising a combination of a honeycombed style insert to a container for fragranced oil;

FIG. 2(A) and 2(B) comprise images of respective different exemplary containers for fragranced oil for use in presently disclosed combinations;

FIG. 3 is an augmented image of an exemplary embodiment of a presently disclosed non-powered diffuser-type device comprising a combination of a honeycombed style insert for a container for fragranced oil;

FIG. 4(A), 4(B), and 4(C) comprise, respectively, an image of a folded embodiment of a topper or insert for use with the exemplary container (jar) of FIG. 2(A), an image of the insert of FIG. 4(A) unfolded, and a side view illustration of the FIG. 4(B) subject matter received in a container of FIG. 2(A), all in accordance with the presently disclosed subject matter;

FIG. 5(A)-5(C) through FIG. 15(A)-15(C) comprise respective images of additional embodiments of a topper or insert for use with the exemplary container (jar) of FIG. 2(A), in folded, unfolded, and combined positions thereof, all in accordance with the presently disclosed subject matter;

FIG. 16(A), 16(B), and 16(C) comprise, respectively, an image of a folded embodiment of a topper or insert for use with the exemplary container (jar) of FIG. 2(B), an image of the insert of FIG. 16(A) unfolded, and a side view illustration of the FIG. 16(B) subject matter received in a container of FIG. 2(B), all in accordance with the presently disclosed subject matter;

FIG. 17(A)-17(C) through FIG. 27(A)-27(C) comprise respective images of additional embodiments of a topper or insert for use with the exemplary container (jar) of FIG. 2(B), in folded, unfolded, and combined positions thereof, all in accordance with the presently disclosed subject matter; and

FIG. 28(A) and 28(B) comprise side elevational views of exemplary embodiments of covers having vertical line design and grid dot design, respectively, for being placed over presently disclosed toppers or inserts and supported on jars or containers associated with the respective toppers, all in accordance with the presently disclosed subject matter.

Repeat use of reference characters in the present specification and drawings is intended to represent the same or analogous features, elements, or steps of the presently disclosed subject matter.

### DETAILED DESCRIPTION OF THE PRESENTLY DISCLOSED SUBJECT MATTER

Reference will now be made in detail to various embodiments of the disclosed subject matter, one or more examples of which are set forth below. Each embodiment is provided by way of explanation of the subject matter, not limitation thereof. In fact, it will be apparent to those skilled in the art that various modifications and variations may be made in the present disclosure without departing from the scope or spirit of the subject matter. For instance, features illustrated or described as part of one embodiment, may be used in another embodiment to yield a still further embodiment.

As used herein, the term “or” is inclusive unless stated otherwise. For instance, if a computer requires A or B to be true in order to perform operation C, the case of both A and B being true will satisfy the condition necessary for C to occur. That is, “or” is inclusive of A, B, and A and B.

In general, the present disclosure is directed to subject matter which relates to improved devices and methodologies for ambient diffuser systems for the dispersion of fragrance. In particular, the present disclosed subject matter involves different embodiments using various combinations of materials and features to provide a flameless and non-powered diffuser-type device providing strong, dependable fragrance dispersion performance.

Some embodiments of the presently disclosed subject matter may relate to foldable honeycomb devices or elements for use in place of reeds for a reed diffuser type of product. Some embodiments of the presently disclosed subject matter may relate to diffusers with origami style foldable paper-based inserts in place of either reeds or sola wood flowers, to achieve effect and efficient ambient diffusion of fragrance.

As used herein, the terms “paper” or “paper-like materials” refer to flexible, substantially planar sheet materials capable of being creased, scored, or folded into macroscopic geometric structures, such as a cellular honeycomb network. This is intended as fundamentally distinct from dense, volumetric matrices formed by tightly winding paper into solid rods or compressing raw pulp fibers into thick, rigid three-dimensional blocks. In the presently disclosed subject matter, the planar nature of the paper-like sheet, combined with its folded honeycomb configuration, provides an exposed macroscopic surface area that maximizes ambient evaporation, rather than relying on the microscopic porosity of a densely compacted pulp mass to slowly release fragrance.

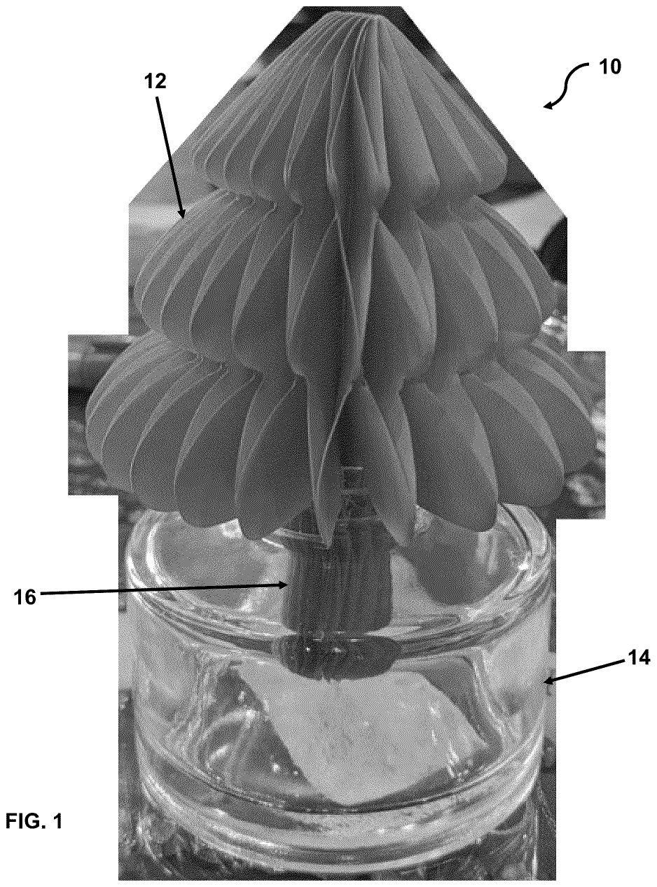

FIG. 1 is an image representing an exemplary embodiment of a presently disclosed non-powered diffuser-type device generally 10. The diffuser-type device comprises a combination of an exemplary foldable honeycombed style insert generally 12 to a container generally 14 for fragranced oil. As discussed herein, insert 12 may be provided with various shapes, both potentially for aesthetic purposes and/or for seasonal decorations. The image of insert 12 of FIG. 1 represents a three-tiered tree outline. The color or colors of materials may also be chosen for decoration and/or aesthetic purposes.

As shown in FIG. 1, the insert 12 has a lower portion generally 16 which has a relatively smaller diameter and extends down into the bottle, for wicking and diffusing the fragrance. Because the honeycomb design may be comprised of paper, and has many folded surfaces, the presently disclosed subject matter provides dramatically increased surface area compared to traditional rattan or fiber reeds, with the resulting diffusion performance and fragrance throw being exceptional.

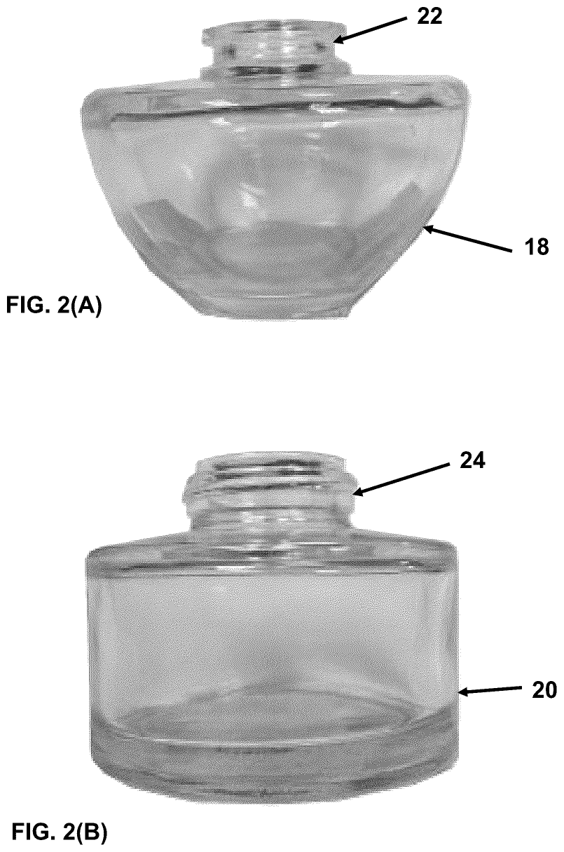

FIG. 2(A) and 2(B) comprise images of respective different exemplary containers 18 and 20 for fragranced oil for use in presently disclosed combinations. While various shapes, sizes, and materials of jars/containers may be practiced, it will be understood from the complete disclosure herewith that the specific inserts may be adapted as needed for proper performance in combination with a particular jar structure. As seen, for example, the respective embodiments of FIG. 2(A) and 2(B) have different length (i.e., height) necks 22 and 24 on their respective upper surfaces, and also have respectively sized openings. Stated another way, even inserts having the same general shape will need to be specifically fitted for use with either of jars 18 or 20 or some other container.

As further discussed herein, a topper or insert element is designed to be packaged in a flat (folded) position, either for commercializing and/or for shipping purposes. When ready for use, it is unfolded by the user into a honeycombed insert, also have a shaped appearance as represented in various present figures.

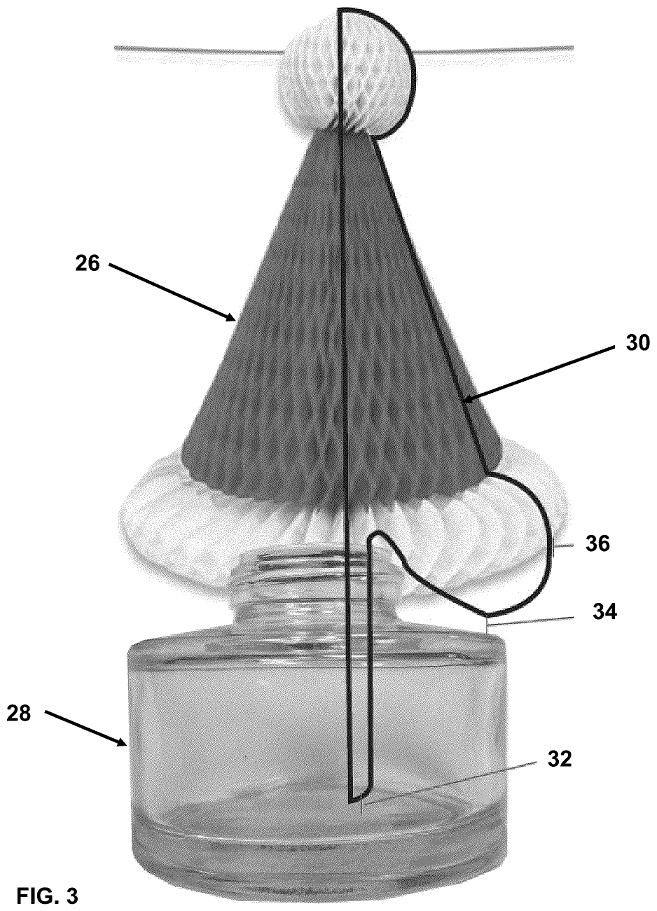

FIG. 3 is an image of an exemplary embodiment of a presently disclosed non-powered diffuser-type device comprising a combination of a honeycombed style insert generally 26 for a container generally 28 for fragranced oil or a fragranced solution. The imagery of FIG. 3 is augmented with a black outline generally 30.

In FIG. 3, insert 26 is unfolded but not yet placed into container 28. Black outline 30 shows locations where various peripheral regions of insert 26 would be located once insert 26 is placed into container 28. Reference number 32 identifies a location at which the bottom of the insert 26 inside the container 28 would be separated above the bottom of the container 28. In some embodiments, such amount of separation could be about ¼ of an inch, in order in a particular combination to have an effective wick or draw action by the insert 26. Reference number 34 similarly identifies a location at which the bottom of the insert 26 outside of the container 28 would be separated above the top side wall of the container 28. Again, In some embodiments, such amount of separation could be about ¼ of an inch. Reference number 36 identifies a section of insert 26 which is the widest part of the insert, and projects laterally out from the upward centerline represented by the vertical component of black outline 30. In some embodiments, such lateral projection may extend outward no more than about ¼ of an inch past the lateral edge of the sidewall of jar 28.

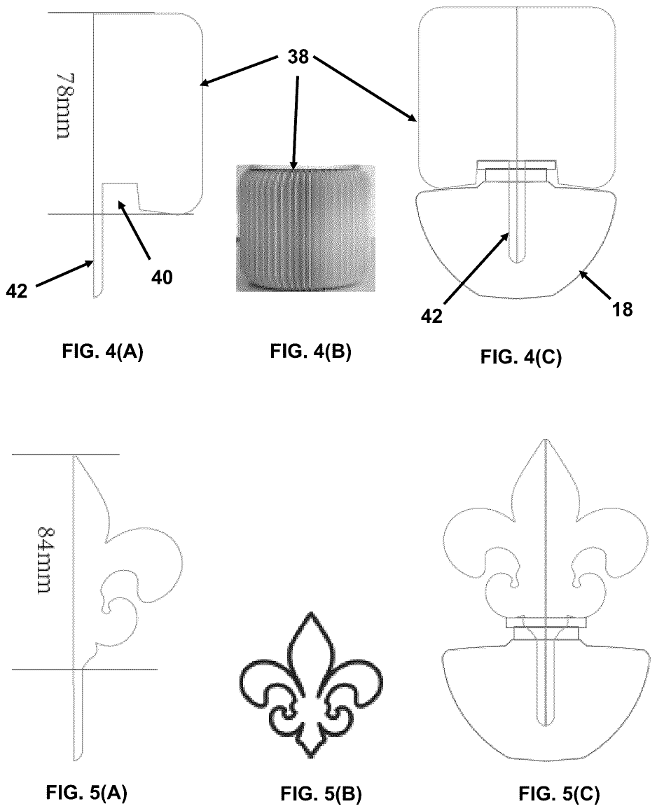

FIG. 4(A) comprises an image of a folded embodiment of a topper or insert generally 38 for use with the exemplary container (jar) of FIG. 2(A). FIG. 4(B) comprises an image of the insert 38 of FIG. 4(A) in an unfolded position thereof. FIG. 4(C) comprises a side view illustration of the FIG. 4(B) subject matter received in a container 18 of FIG. 2(A), all in accordance with the presently disclosed subject matter. It is to be understood from the complete disclosure herewith that insert 38 may be used in combination with other jars or containers other than in the form of exemplary container 18, and adapted for such use as needed.

It should be understood that for many embodiments of presently disclosed subject matter, unfolding means in effect opening the folded flat insert by using a circular motion about the long axis of the insert. For example, in FIG. 4(A), the long axis is the length which runs along the marked 78 mm distance, and then continues along a lower extending region generally 42 (discussed further below). This means that the insert “fans” out as represented in FIG. 4(B) as it unfolds.

It is also to be understood that a pair of cooperating elements (not shown) of various designs may be practiced for holding insert 38 in an unfolded position. For example, cooperating hook and loop elements may be practiced in respective positions that are brought adjacent to each other whenever the insert 38 is unfolded. Other elements or devices may be practiced. For example, snaps or equivalents may be used, or self-sticking portions of tape or similar used, or cooperating magnets. In the case of magnets, the magnets may be coated to prevent any reaction between metal of the magnets and the essence of the fragrance oil materials.

Also, for shipping purposes, the fragrance oil materials may be in a separate container for shipment or may be shipped in jar 18, which is otherwise sealed with a lid (for example, a screw-on or cork type top) which is removed before use with the insert 38. When shipped in a separate container, it will be understood that the fragrance oil or solution will be poured from a separate container into the jar or container 18 prior to its use with the insert 38.

As seen in FIG. 4(A), insert 38 may have a height of about 78 mm above the receiving jar 18. A recessive section generally 40 is incorporated into the design to provide a proper arrangement in accordance with presently disclosed subject matter relative to the jar 18, for proper wicking and support of the insert. In some designs of insert 38 which may be practiced, the section 40 which appears as a recess specifically in FIG. 4(A) may simply be an unoccupied region or area of the insert design. However, in all designs, a lower extending region generally 42 provides wicking functionality as otherwise discussed herein. It will be understood by those of ordinary skill in the art from the complete disclosure herewith that lower extending region 42 appears in its folded position in FIG. 4(A) while appearing in its unfolded position in FIG. 4(C).

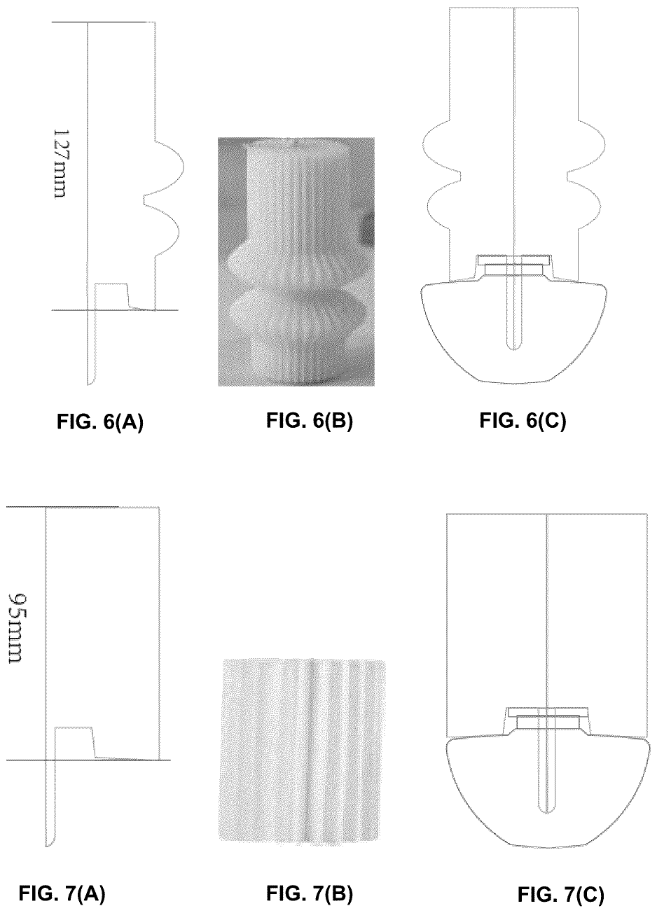

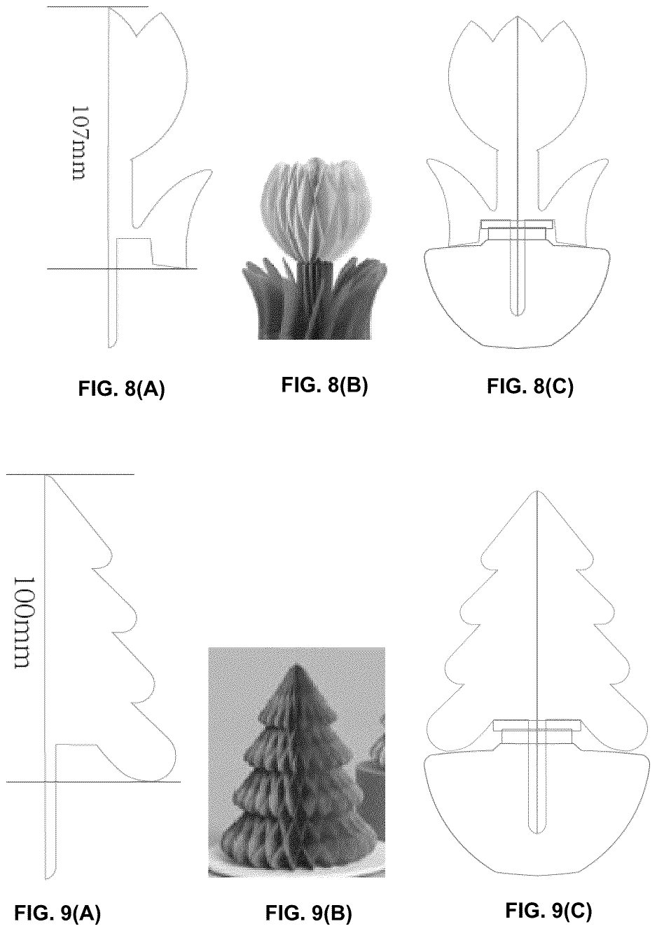

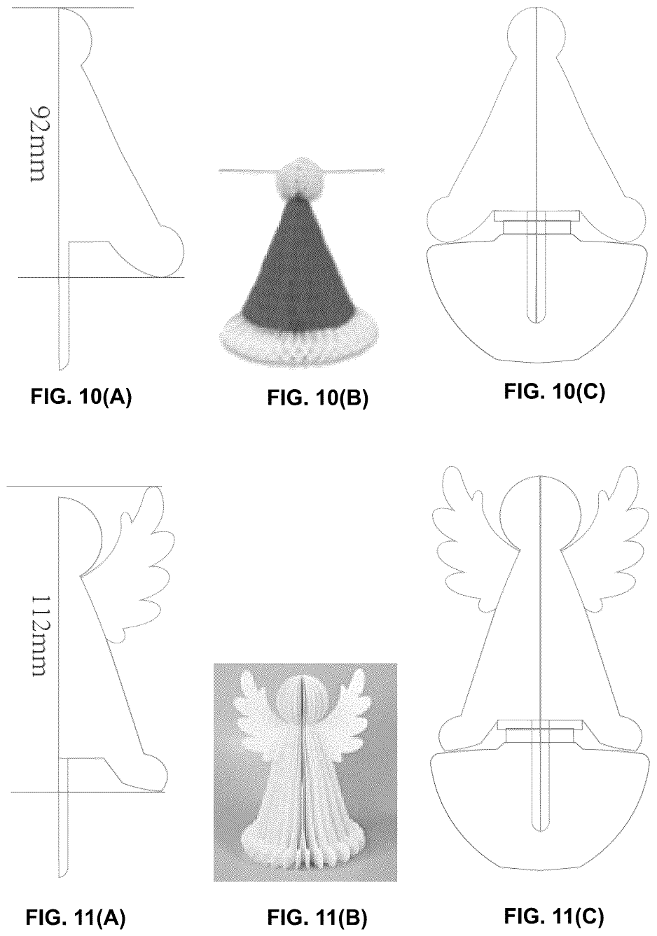

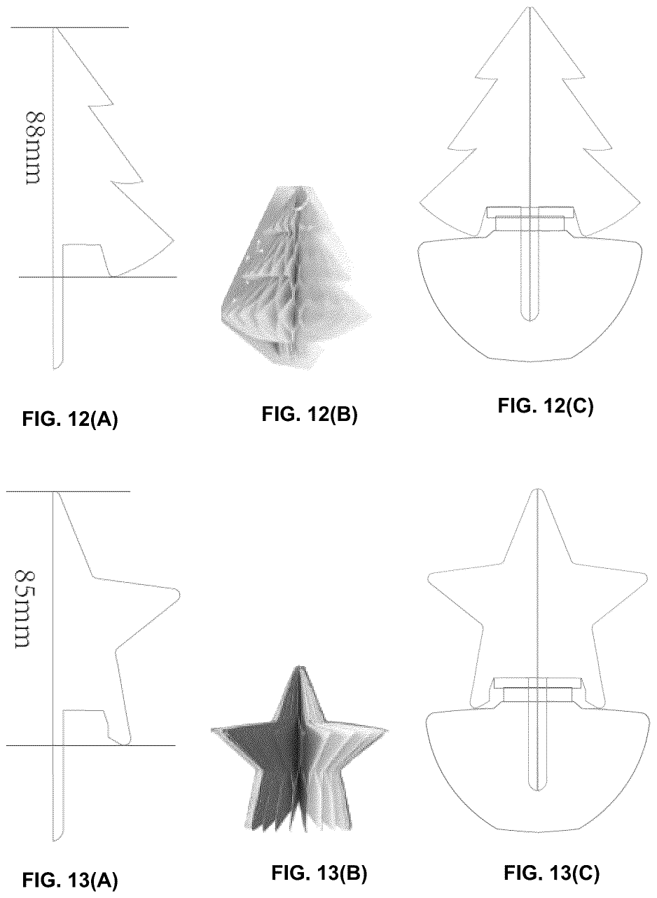

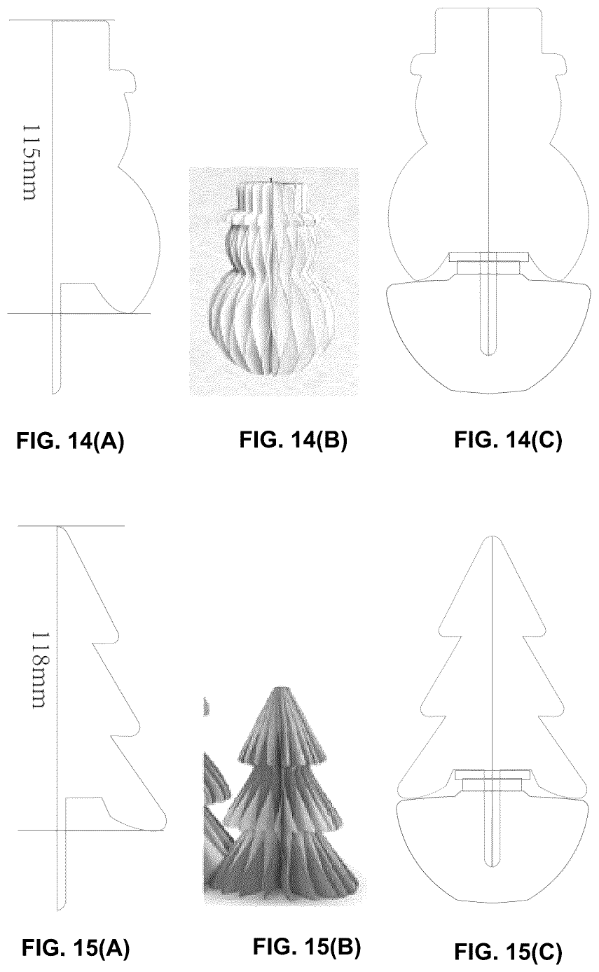

FIG. 5(A)-5(C) through FIG. 15(A)-15(C) comprise respective images of additional embodiments of a topper or insert for use with the exemplary container (jar) of FIG. 2(A), in folded, unfolded, and combined positions thereof, all in accordance with the presently disclosed subject matter.

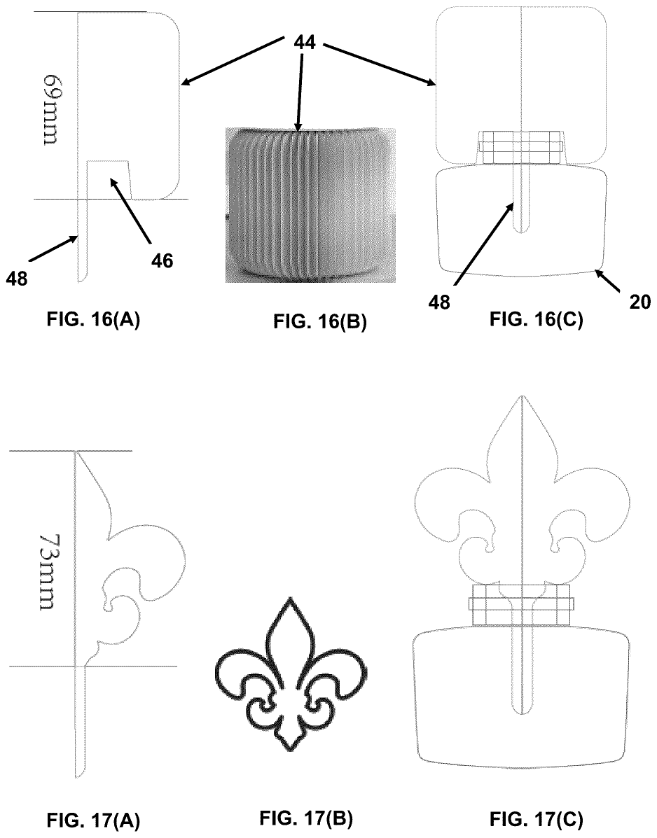

FIG. 16(A) comprises an image of a folded embodiment of a topper or insert generally 44 for use with the exemplary container (jar) 20 of FIG. 2(B). FIG. 16(B) comprises an image of the insert 44 of FIG. 16(A) in an unfolded position thereof. FIG. 16(C) comprises a side view illustration of the FIG. 16(B) subject matter received in a container 20 of FIG. 2(B), all in accordance with the presently disclosed subject matter.

As seen in FIG. 16(A), insert 44 may have a height of about 69 mm above the receiving jar 20. A recessive section generally 46 is incorporated into the design to provide a proper arrangement in accordance with presently disclosed subject matter relative to the jar 20, for proper wicking and support of the insert. In all designs, a lower extending region generally 48 provides wicking functionality as otherwise discussed herein. It will be understood by those of ordinary skill in the art from the complete disclosure herewith that lower extending region 48 appears in its folded position in FIG. 16(A) while appearing in its unfolded position in FIG. 16(C).

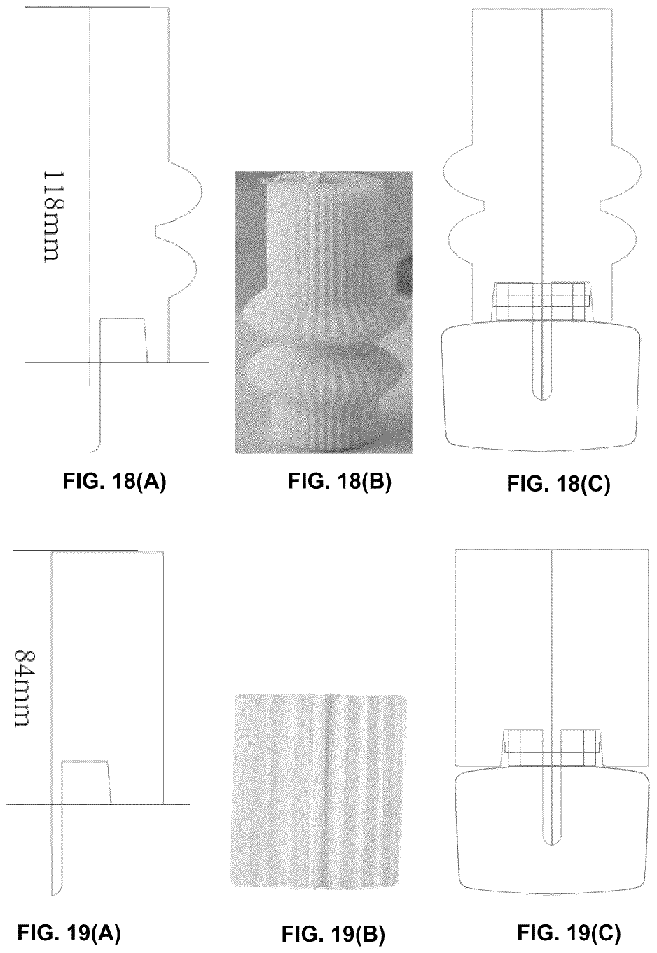

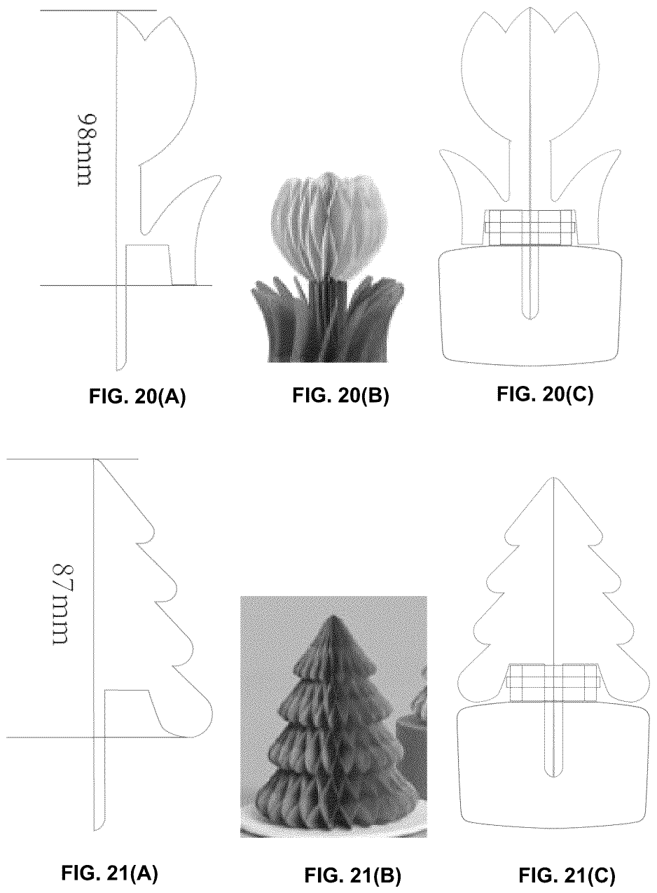

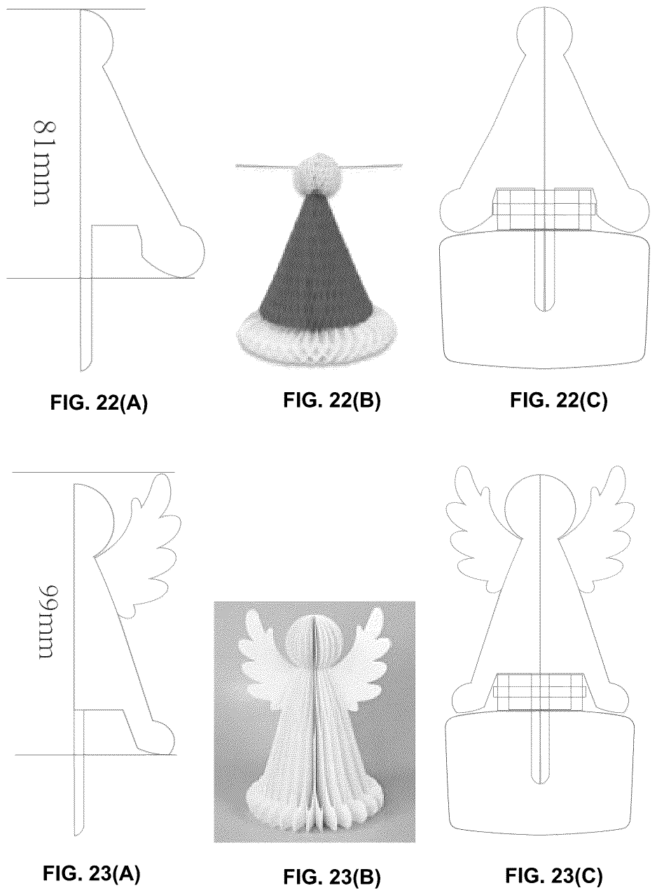

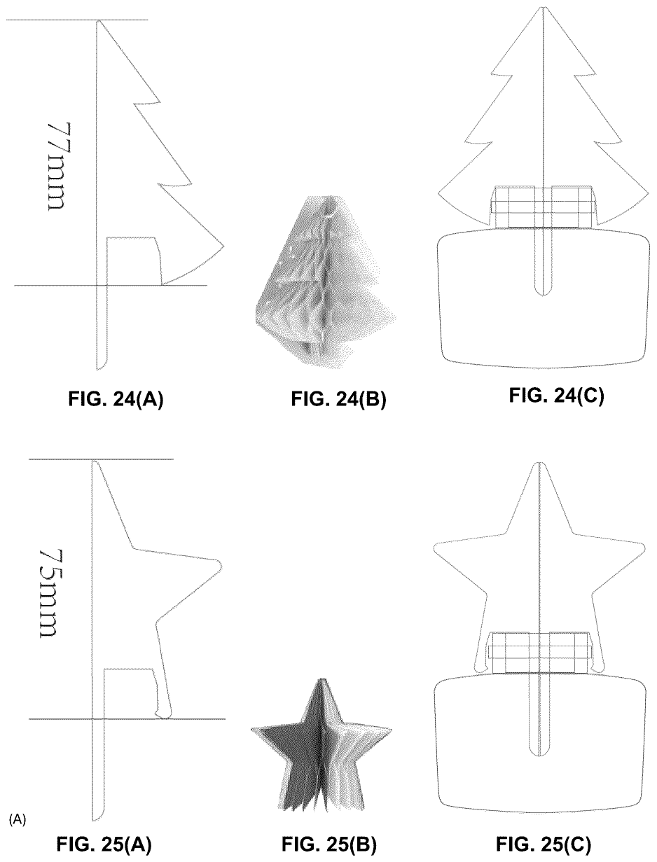

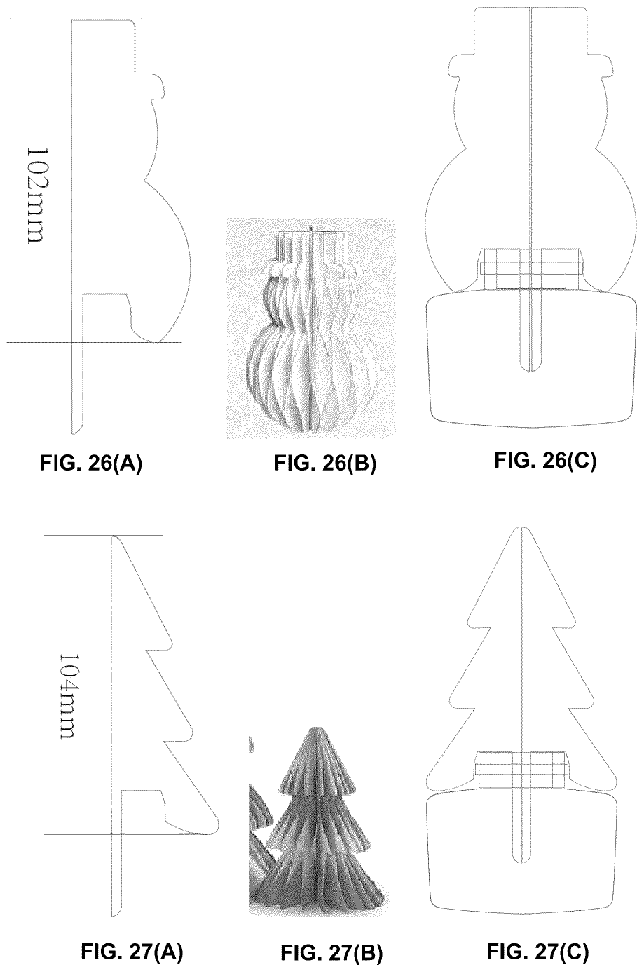

FIG. 17(A)-17(C) through FIG. 27(A)-27(C) comprise respective images of additional embodiments of a topper or insert for use with the exemplary container (jar) 20 of FIG. 2(B), in folded, unfolded, and combined positions thereof, all in accordance with the presently disclosed subject matter.

It may be seen in all of the FIG. (A)'s associated with FIG. 5(A) though 27(A) that exemplary height designations in mm are shown for the portion of the insert above the lower extending portion. Similarly, discussion and description of each of the respective FIGS. (A), (B) and (C) of FIG. 5(A) through 27(C) are all similar for corresponding features and elements.

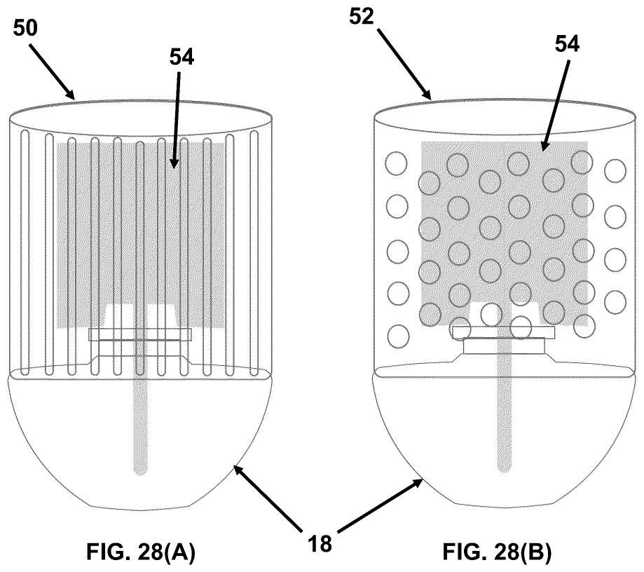

FIG. 28(A) and 28(B) comprise side elevational views of exemplary embodiments of covers 50 and 52 having vertical line design and grid dot design, respectively, for being placed over presently disclosed exemplary toppers or inserts 54 and supported on exemplary jars or containers 18 associated with the respective toppers, all in accordance with the presently disclosed subject matter. As shown, the respective bottoms of covers 50 and 52 may rest on upper facing side wall areas of jars 18. In addition to any affects the presence of covers 50 and 52 may have on the dispersal patterns of fragrance from inserts 54, covers 50 and 52 may also provide various decorative and/or aesthetic purposes. Similarly, the jars may be provided with various decorative and/or aesthetic finish options, including for example, but not limited to, matte spray finish on outside, champagne color mercury outside, champagne color mercury outside with matte finish spray over, and heavy reflective iridescent spray outside.

By way of specific example, and to further illustrate the functional geometry of some embodiments of the presently disclosed subject matter, an exemplary non-powered diffuser device may be assembled and deployed by a user. A deployable insert, formed of a paper or paper-like material, is initially provided in a packaged folded flat position. A user transitions the deployable insert into a deployed fanned-out position by applying a motion (in some instances, a circular motion) about the longitudinal axis of the insert, thereby expanding an upper diffusing region radially outward. For some embodiments, the deployable insert may be secured in this deployed fanned-out position using a pair of cooperating elements, such as magnetic closures, hook and loop fasteners, or adhesive strips.

Once expanded, a lower extending region of the deployable insert is inserted downwardly through an opening of a container holding a fragranced oil or a fragranced solution. For some embodiments, the insert is lowered until a recessive section (such as comprising an unoccupied geometric region formed along the periphery of the insert) receives and rests upon an upper portion or extended neck of the container. In this seated position, the recessive section structurally supports the expanded upper diffusing region above the container, while simultaneously suspending the lower extending region within the fragranced oil, maintaining a predetermined wicking clearance above the bottom of the container. Through capillary action, the fragranced oil is drawn upwardly through the lower extending region and into the honeycombed structure of the upper diffusing region, where the plurality of folded surfaces provides a drastically expanded surface area for highly efficient ambient evaporation and scent dispersion.

As shown by the foregoing, the presently disclosed subject matter utilizes structural geometry (particularly macro-geometry) to achieve highly effective non-powered, flameless dispersion of fragrance. By employing a foldable honeycombed style structure that actively wicks from a reservoir of fragranced oil or a fragranced solution, the presently disclosed devices and corresponding methodologies are capable of regulating and maintaining optimal fragrance dispersion. Consequently, the presently disclosed devices and methods can achieve superior performance relying primarily on functional geometry, without strictly requiring the use of complex chemical coatings, heavily engineered layers of diverse pulp or fiber types, or artificially created micro-pores to regulate fragrance release.

Accordingly, the structural configurations of the presently disclosed deployable inserts provide several objective technical effects that solve technical problems associated with conventional diffusion systems. Specifically, the foldable honeycombed macro-geometry provides the technical effect of maximizing evaporative surface area while decoupling the wicking mechanism from rigid microscopic pores, thereby solving the technical problem of wicking pathways becoming partially loaded or blocked over time with heavier molecular weight aromatic materials. Furthermore, the structural capability of the insert to transition from a packaged folded flat position to a deployed fanned-out position provides the technical effect of drastically reducing required commercial packaging and shipping volume compared to rigid diffusion elements. Additionally, the mechanical geometry of the insert, particularly the provision of the recessive section, provides the technical effect of dynamically adapting to and securely supporting a top-heavy expanded diffusing region over containers of varying neck heights, safely suspending the lower wicking region within the fragranced oil. These technical features synergistically interact to provide the unexpected technical advantage of a highly efficient, non-powered, and dependable ambient fragrance dispersion system that is both commercially scalable and mechanically stable.

This written description uses examples to disclose the presently disclosed subject matter, including the best mode, and also to enable any person skilled in the art to practice the presently disclosed subject matter, including making and using any devices or systems and performing any incorporated methods. The patentable scope of the presently disclosed subject matter is defined by the claims, and may include other examples that occur to those skilled in the art. Such other examples are intended to be within the scope of the claims if they include structural and/or step elements that do not differ from the literal language of the claims, or if they include equivalent structural and/or elements with insubstantial differences from the literal languages of the claims. In any event, while certain embodiments of the disclosed subject matter have been described using specific terms, such description is for illustrative purposes only, and it is to be understood that changes and variations may be made without departing from the spirit or scope of the subject matter. Also, for purposes of the present disclosure, the terms “a” or “an” entity or object refers to one or more of such entity or object. Accordingly, the terms “a”, “an”, “one or more,” and “at least one” can be used interchangeably herein.

What is claimed is:

1. A non-powered diffuser device for the ambient dispersion of fragrance, comprising:
a container defining an interior volume holding a fragranced oil or a fragranced solution, the container having an upper portion defining an opening; and
a deployable insert positioned at least partially within the container, the deployable insert comprising a foldable honeycombed style body formed from a paper or paper-like material and configured to transition from a packaged folded flat position into a deployed fanned-out position;
wherein in the deployed fanned-out position, the deployable insert comprises:
a lower extending region projecting downwardly through the opening and into the fragranced oil or a fragranced solution within the container interior volume to draw or wick the fragranced oil or a fragranced solution via capillary action; and
an upper diffusing region expanding radially outward relative to a longitudinal axis of the deployable insert, the upper diffusing region comprising a plurality of folded surfaces providing an expanded surface area for ambient evaporation of the wicked fragranced oil or a fragranced solution.

2. The non-powered diffuser device according to claim 1, wherein the deployable insert is configured to transition to the deployed fanned-out position via a circular motion about the longitudinal axis.

3. The non-powered diffuser device according to claim 1, further comprising a pair of cooperating elements configured to hold the deployable insert in the deployed fanned-out position.

4. The non-powered diffuser device according to claim 3, wherein the pair of cooperating elements comprise at least one of hook and loop elements, snaps, self-sticking tape, and coated magnets.

5. The non-powered diffuser device according to claim 1, wherein a bottom edge of the lower extending region is separated from the bottom of the interior volume of the container by a predetermined wicking clearance.

6. The non-powered diffuser device according to claim 1, further comprising:
a recessive section formed along a periphery of the deployable insert between the lower extending region and the upper diffusing region, the recessive section defining an unoccupied geometric region shaped to receive the upper portion of the container, whereby the recessive section seats the upper diffusing region above the container and suspends the lower extending region above a bottom of the interior volume; and
wherein
a lower edge of the upper diffusing region is separated from a top side wall of the container by a separation distance to facilitate the ambient evaporation.

7. The non-powered diffuser device according to claim 6, wherein the upper portion of the container comprises an extended neck, and wherein the recessive section of the deployable insert is dimensioned to rest upon the extended neck.

8. The non-powered diffuser device according to claim 1, wherein the upper diffusing region comprises a lateral projection extending outward past a lateral edge of a sidewall of the container.

9. The non-powered diffuser device according to claim 1, wherein:
the container comprises a decorative finish, and
the upper diffusing region of the deployable insert comprises a shaped appearance and at least one chosen color to provide a decorative appearance in the deployed fanned-out position.

10. A method for providing ambient dispersion of a fragrance, comprising:
providing a container holding a fragranced oil or a fragranced solution, the container having an upper portion defining an opening;
providing a deployable insert in a packaged folded flat position, the deployable insert comprising a foldable honeycombed style body formed from a paper or paper-like material;
unfolding the deployable insert from the packaged folded flat position into a deployed fanned-out position;
positioning the deployable insert at least partially into the container such that a lower extending region of the deployable insert projects downwardly through the opening and into the fragranced oil or a fragranced solution, while an upper diffusing region of the deployable insert projects upwardly above the opening and provides an expanded surface area of folded surfaces for ambient evaporation of the fragranced oil or a fragranced solution; and
drawing or wicking the fragranced oil or a fragranced solution upward through the lower extending region and into the upper diffusing region through capillary action.

11. The method according to claim 10, wherein unfolding the deployable insert comprises using a circular motion about a long axis of the deployable insert.

12. The method according to claim 10, further comprising the step of securing the deployable insert in the deployed fanned-out position using a pair of cooperating elements prior to positioning the deployable insert into the container.

13. The method according to claim 10, further comprising:
providing the deployable insert a recessive section which receives the upper portion of the container to support an upper diffusing region of the deployable insert above the container; and
removing a sealed lid from the container holding the fragranced oil or a fragranced solution prior to positioning the deployable insert into the container.

14. The method according to claim 10, further comprising the step of pouring the fragranced oil or a fragranced solution from a separate shipping container into the container prior to positioning the deployable insert into the container.

15. The method according to claim 10, wherein the container comprises a decorative finish and the upper diffusing region of the deployable insert comprises a shaped appearance and at least one chosen color to provide a decorative appearance in the deployed fanned-out position.

16. A non-powered diffuser device for the ambient dispersion of fragrance, comprising:
a container defining an interior volume holding a fragranced oil or a fragranced solution, the container having an upper facing side wall area and defining an opening;
an insert comprising a lower wicking region received through the opening into the interior volume to draw or wick the fragranced oil or a fragranced solution via capillary action, and an upper diffusing region extending above the container, the insert comprising a foldable honeycombed style body formed from a paper or paper-like material; and
a cover positioned over the upper diffusing region of the insert, the cover having a bottom resting on the upper facing side wall area of the container;
wherein the cover defines a plurality of openings permitting ambient dispersion of the fragranced oil or a fragranced solution evaporated from the upper diffusing region.

17. The non-powered diffuser device according to claim 16, wherein the plurality of openings in the cover comprise a vertical line design.

18. The non-powered diffuser device according to claim 16, wherein the plurality of openings in the cover comprise a grid dot design.

19. The non-powered diffuser device according to claim 16, wherein the insert is configured to transition between a folded position for packaging and an unfolded position for use.

20. The non-powered diffuser device according to claim 19, wherein the container comprises a decorative finish, and the upper diffusing region of the insert comprises a shaped appearance and at least one chosen color to provide a decorative appearance in an unfolded position.
